(19) United States
(12) Patent Application Publication
(10) Pub. No.: US 2025/0228984 A1
(43) Pub. Date: Jul. 17, 2025

(54) Metallophore-Radionuclide Complex as Nuclear Imaging Contrast Agents

(71) Applicant: University of Cincinnati, Cincinnati, OH (US)

(72) Inventors: Nalinikanth Kotagiri, West Chester, OH (US); Alec Ventrola, Liberty Township, OH (US)

(73) Assignee: University of Cincinnati, Cincinnati, OH (US)

(21) Appl. No.: 19/018,908

(22) Filed: Jan. 13, 2025

Related U.S. Application Data
(60) Provisional application No. 63/619,758, filed on Jan. 11, 2024.

(51) Int. Cl. A61K 51/04

(52) U.S. Cl. CPC A61K 51/0478; A61K 51/0482; A61K 2123/00

(57) ABSTRACT

The present invention involves a method of targeted imaging of a bacterial infection in a subject. In one embodiment, the method includes administering pyochelin (PcH) bound to a radiometal to the subject and imaging the infected area of the subject using a positron emission tomography-computed tomography (PET/CT) scan. In one embodiment, the pyochelin bound to a radiometal is selected from the group consisting of copper-64 bound pyochelin and gallium-68 bound pyochelin.

### CROSS REFERENCE TO RELATED APPLICATIONS

This application claims priority to U.S. Provisional Application Ser. No. 63/619,758, filed Jan. 11, 2024, which application is hereby incorporated by reference in its entirety.

### STATEMENT REGARDING FEDERALLY FUNDED RESEARCH OR DEVELOPMENT

This invention was made with government support under HL168588 awarded by the National Institutes of Health. The government has certain rights in the invention.

### TECHNICAL FIELD

The present invention relates to the development of a nuclear imaging contrast agent for imaging bacterial infections.

### BACKGROUND OF THE INVENTION

Pseudomonas aeruginosa is a pathogenic bacterium that causes severe lower respiratory infections, most notably in cases of chronic lung conditions like cystic fibrosis or chronic obstructive pulmonary disease (COPD). It can be difficult to fully image an infection of this bacterium in the lungs or other organs. Therefore, an improved method is needed to image the bacterium in patients.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

The present invention involves a method of targeted imaging of a bacterial infection in a subject. In one embodiment, the method includes administering pyochelin (PcH) bound to a radiometal to the subject and imaging the infected area of the subject using a positron emission tomography-computed tomography (PET/CT) scan. In one embodiment, the pyochelin bound to a radiometal is selected from the group consisting of copper-64 bound pyochelin and gallium-68 bound pyochelin. In another embodiment, the pyochelin bound to a radiometal is copper-64 bound pyochelin. In one embodiment, the pyochelin is bound to copper-64 in a reaction at a pH between 5.5 and 7. In another embodiment, the pyochelin bound to a radiometal is gallium-68 bound pyochelin. In one embodiment, the pyochelin is bound to gallium-68 in a reaction at a pH between 5.5 and 7. In another embodiment, the bacterial infection comprises the bacteria Pseudomonas aeruginosa.

The present invention further involves an additional method of targeted imaging of a bacterial infection in a subject. In this embodiment, the method includes administering desferrioxamine B (DFO) bound to a radiometal to the subject and imaging the infected area of the subject using a positron emission tomography-computed tomography (PET/CT) scan.

In one embodiment, the DFO bound to a radiometal is selected from the group consisting of copper-64 bound DFO and gallium-68 bound DFO. In another embodiment, the DFO bound to a radiometal is gallium-68 bound DFO. In one embodiment, the DFO is bound to gallium-68 in a reaction at a pH between 3 and 7. In another embodiment, the DFO is bound to gallium-68 in a reaction at a pH between 3 and 5.5. In one embodiment, the DFO bound to a radiometal is copper-64 bound DFO. In another embodiment, the DFO is bound to copper-64 in a reaction at a pH between 3 and 7. In one embodiment, the bacterial infection comprises the bacteria Pseudomonas aeruginosa.

### BRIEF DESCRIPTION OF THE DRAWINGS

The foregoing summary, as well as the following detailed description of preferred embodiments of the application, will be better understood when read in conjunction with the appended drawings.

FIG. 1A is a graph showing Cu-64-Pch stability at various time points after incubation.

FIG. 1B is a graph showing a Radio chromatogram of Cu-64-Pch prepared at pH 7.

FIG. 1C is a graph showing a radio chromatogram of Cu-64-Pch at a Pch concentration of 0.5 mL.

FIG. 1D is a graph showing a radio chromatogram of Cu-64-Pch at a Pch concentration of 1.0 mL.

FIG. 1E is a graph showing a radio chromatogram of Cu-64-Pch at a Pch concentration of 1.5 mL.

FIG. 1F is a graph showing a radio chromatogram of Cu-64-Pch at a Pch concentration of 2.0 mL.

FIG. 1G is a graph showing a radio chromatogram of Cu-64-Pch at a Pch concentration of 4.0 mL.

FIG. 2A is a graph showing a radio chromatogram of Cu-64-Pch at an incubation time of 10 minutes.

FIG. 2B is a graph showing a radio chromatogram of Cu-64-Pch at an incubation time of 20 minutes.

FIG. 2C is a graph showing a radio chromatogram of Cu-64-Pch at an incubation time of 30 minutes.

FIG. 2D is a graph showing a radio chromatogram of Cu-64-Pch at an incubation time of 45 minutes.

FIG. 2E is a graph showing a radio chromatogram of Cu-64-Pch at an incubation time of 60 minutes.

FIG. 3A is a graph showing a UV chromatogram of ammonium acetate (Amm. Ace.) with no Pch.

FIG. 3B is a graph showing a UV chromatogram of Pch at a concentration of 25 μL PcH+325 μL Amm. Ace.

FIG. 3C is a graph showing a UV chromatogram of Pch at a concentration of 50 μL PcH+300 μL Amm. Ace.

FIG. 3D is a graph showing a UV chromatogram of Pch at a concentration of 100 μL PcH+200 μL Amm. Ace.

FIG. 3E is a graph showing a UV chromatogram of Pch at a concentration of 200 μL PcH+150 μL Amm. Ace.

FIG. 4A is a graph showing a TLC diagram of Lead-203.

FIG. 4B is a graph showing a TLC diagram of Copper-64.

FIG. 4C is a graph showing a TLC diagram of Zirconium-89.

FIG. 4D is a graph showing a TLC diagram of Titanium-45.

FIG. 4E is a graph showing a TLC diagram of Manganese-52.

FIG. 4F is a graph showing a TLC diagram of Gallium-68.

FIG. 4G is a graph showing radio chromatograms of free gallium-68 metal, pyochelin, gallium-68 bound pyochelin, gallium-68 bound pyochelin with DTPA competitor, and gallium-68 bound pyochelin in serum.

FIG. 5A is a PET/CT image of saline.

FIG. 5B is a PET/CT image of K. pneumoniae.

FIG. 5C is a PET/CT image of P. aeruginosa non-mucoid.

FIG. 5D is a PET/CT image of P. aeruginosa mucoid.

FIG. 5E is a PET/CT image of E. coli Nissle.

FIG. 5F is a PET/CT image of E. coli UT189.

FIG. 5G is a PET/CT image of S. aureus.

FIG. 5H is a graph showing biodistribution of 64Cu-PcH in the lungs.

FIG. 5I is a PET/CT image of intra-tracheally administered PAOI.

FIG. 5J is a PET/CT image of intra-tracheally administered PAOI.

FIG. 5K is a PET/CT image of intra-tracheally administered saline control.

FIG. 5L is a graph showing biodistribution of PAOl administered intra-tracheally vs. PAOI administered intra-nasally vs. saline.

FIG. 6A is a pair of PET images for PA ATCC 15692.

FIG. 6B is a pair of PET images for 0.9% saline.

FIG. 6C is a pair of PET images for KP ATCC 70021.

FIG. 6D is a graph showing biodistribution of 68Ga-Pch for the heart, lungs, liver, kidneys.

FIG. 6E is a graph showing biodistribution of 68Ga-Pch for the lungs.

FIG. 7A is a pair of PET images for PA ATCC 15692.

FIG. 7B is a pair of PET images for 0.9% saline.

FIG. 7C is a pair of PET images for KP ATCC 70021.

FIG. 7D is a graph showing biodistribution of 68Ga-DFO for the heart, lungs, liver, kidneys.

FIG. 7E is a graph showing biodistribution of 68Ga-DFO for the lungs.

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Pseudomonas aeruginosa is a pathogenic bacterium that causes severe lower respiratory infections, most notably in cases of chronic lung conditions like cystic fibrosis or chronic obstructive pulmonary disease (COPD). This organism, along with other bacteria, requires metals to perform basic metabolic processes to survive. P. aeruginosa produces two different siderophores, pyochelin (Pch) and pyoverdine. These molecules are responsible for gathering iron from the extracellular environment and transporting it to a specific cell surface receptor, fptA, found exclusively on P. aeruginosa bacterial cells. These siderophores have the ability to bind to free metals in the environment. The present invention uses this mechanism to bind the siderophore with a radiometal to allow for in vivo applications such as targeted imaging of the infection, specifically within the lungs in chronic or complex cases.

The present invention binds a siderophore such as pyochelin (associated with fptA) or desferrioxamine B (hereinafter DFO), which is associated with FoxA, to either copper-64 (Cu-64) or Gallium (Ga-68) radiometals. The complexes are used as nuclear imaging contrast agents in a PET/CT scan.

Positron emission tomography-computed tomography (PET/CT) is a nuclear medicine technique which combines, in a single gantry, a positron emission tomography (PET) scanner and an x-ray computed tomography (CT) scanner, to acquire sequential images from both devices in the same session, which are combined into a single superposed (co-registered) image. Thus, functional imaging obtained by PET, which depicts the spatial distribution of metabolic or biochemical activity in the body can be more precisely aligned or correlated with anatomic imaging obtained by CT scanning.

The siderophores were evaluated in their ability to bind to copper-64 (Cu-64) and Gallium (Ga-68) radiometals using high-performance liquid chromatography. Furthermore, in vivo imaging studies were performed to investigate the quality and selectivity of both the Cu-64 or Ga-68 bound pyochelin and the Ga-68 bound DFO, and their ability to provide insight into the complex infections within the lungs.

The data presented for the in vitro studies described herein indicate pyochelin complexes extremely well with copper-64. It is also seen in the in vivo images that Cu-64-Pch is transported into the P. aeruginosa cells and is not transported by bacterial cells that don't express the fptA receptor. Biodistribution also shows elevated levels in the P. aeruginosa containing lungs. The biodistribution also displays that intra-tracheal administration is superior to intra-nasal administration and leads to a larger number of bacterial colonies that localize in the lungs as opposed to intra-nasal method where bacteria may localize in the upper respiratory tract, esophagus, mouth, or stomach.

The data shows high binding affinity between Cu-64 and PcH at both pH 5.5 and pH 7 and at 37° C. (Table 1).

TABLE 1
Preliminary screen of Cu-64 with different siderophores.

| | | | | pH | pH | pH | pH |
|---|---|---|---|---|---|---|---|
| Radiometal | Siderophore | Temp | Time | 3 | 5.5 | 7.0 | 9 |
| ⁶⁴Cu | BcB | 37° C. | 60 mins | 5 | 8 | 13 | 15 |
| ⁶⁴Cu | BcB | 90° C. | 30 mins | 10 | 18 | 15 | 11 |
| ⁶⁴Cu | PcH | 37° C. | 60 mins | 0 | 85 | 95 | 0 |
| ⁶⁴Cu | PcH | 90° C. | 30 mins | 0 | 0 | 0 | 0 |
| ⁶⁴Cu | SmC | 37° C. | 60 mins | 0 | 10 | 40 | 0 |
| ⁶⁴Cu | SmC | 90° C. | 30 mins | 5 | 0 | 0 | 28 |
| ⁶⁴Cu | StP | 37° C. | 60 mins | 0 | 0 | 7 | 15 |
| ⁶⁴Cu | StP | 90° C. | 30 mins | 0 | 0 | 1 | 3 |
| ⁶⁴Cu | YbT | 37° C. | 60 mins | 0 | 85 | 95 | 10 |
| ⁶⁴Cu | YbT | 90° C. | 30 mins | 0 | 47 | 70 | 56 |

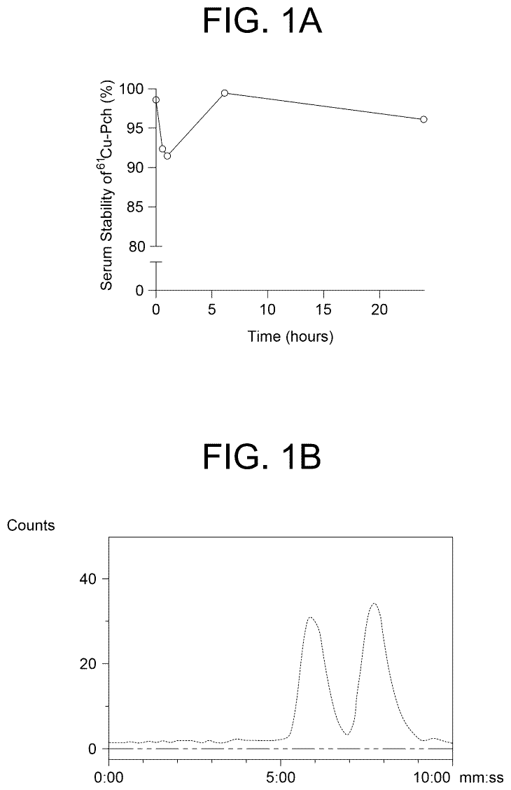

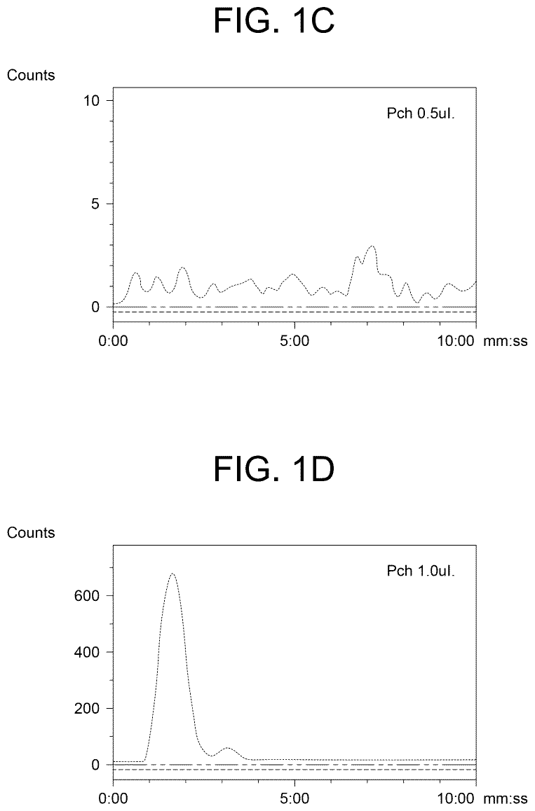

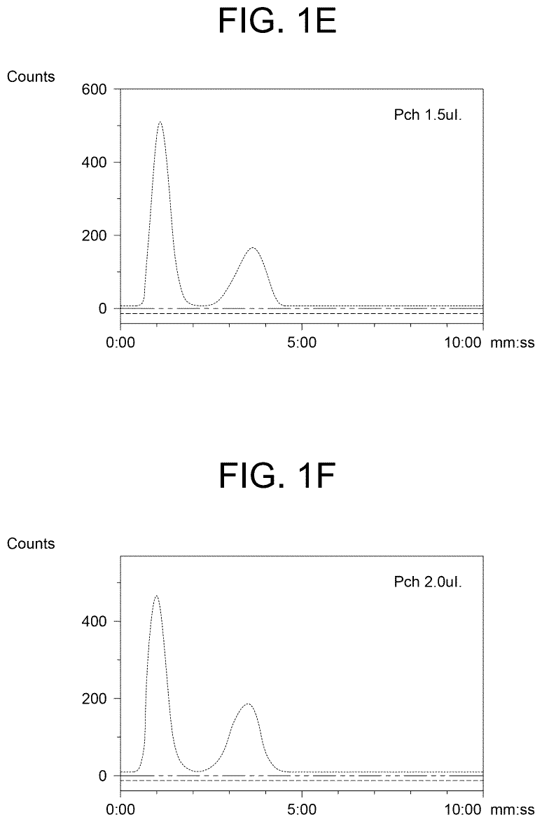

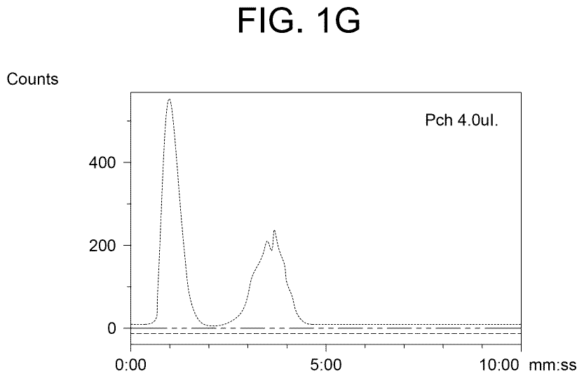

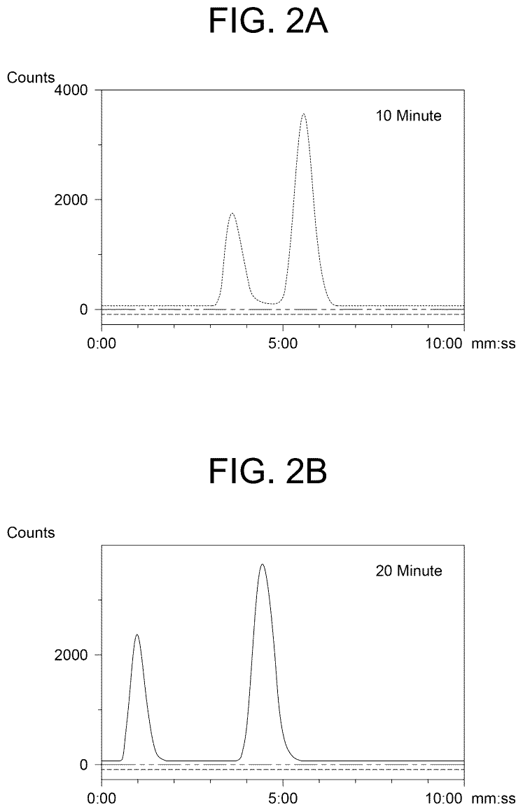

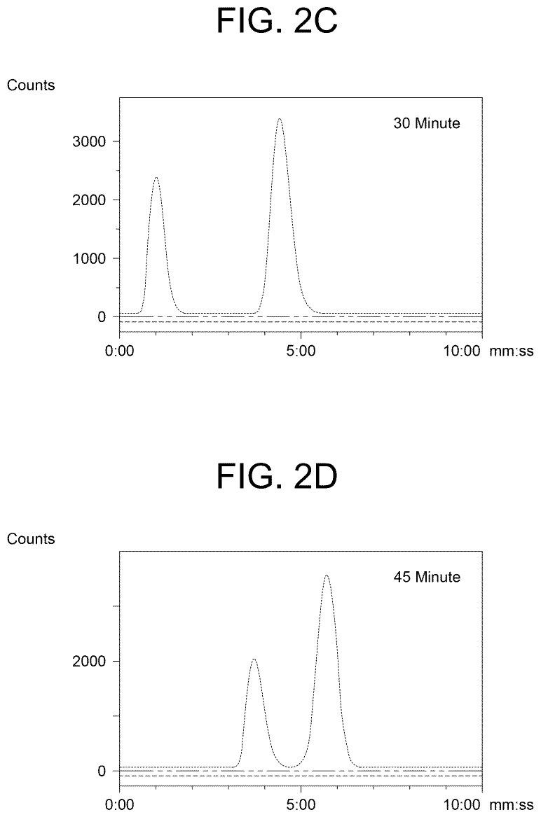

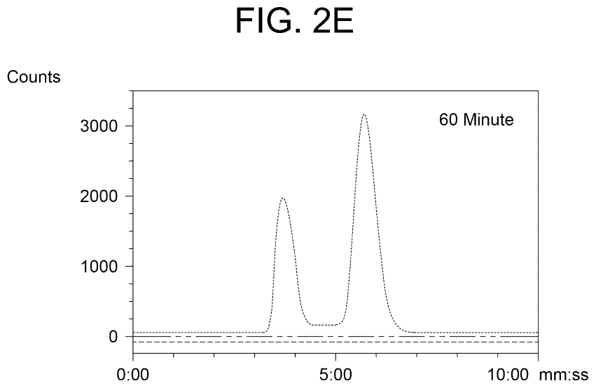

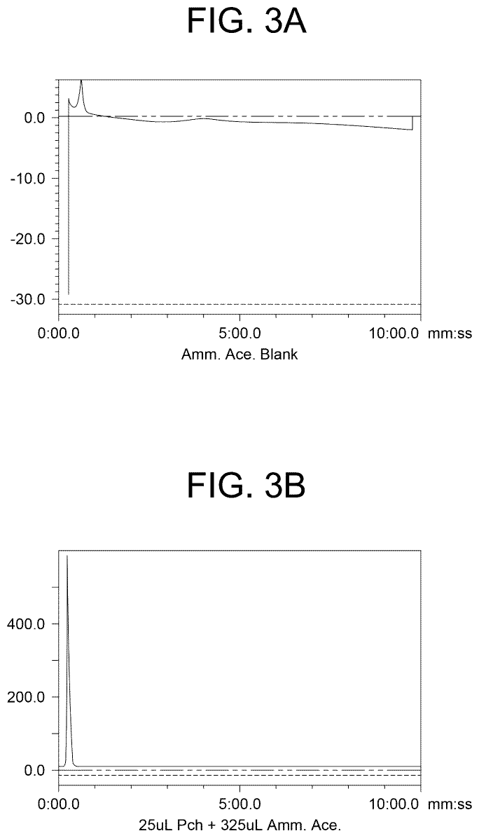

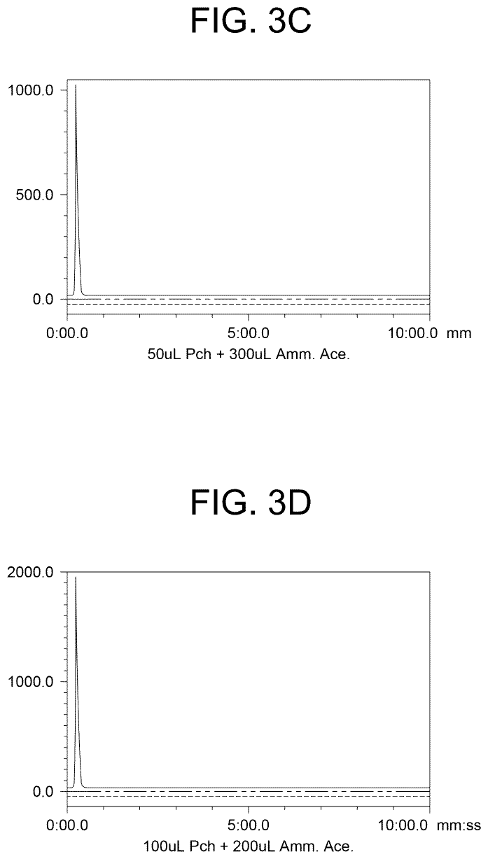

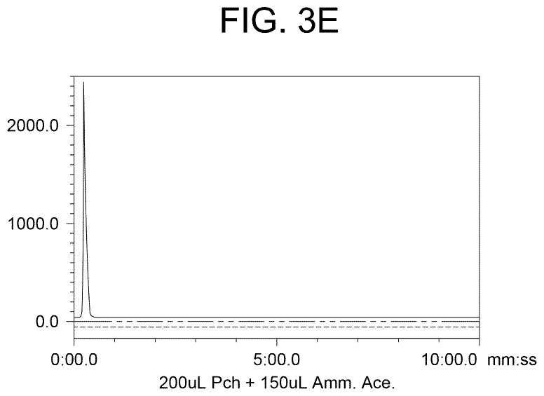

HPLC stability studies of complexed Cu-64-Pch show a minimum of 91% stability after 24 hours of serum incubation (FIG. 1A). Furthermore, initial HPLC purity data shows two peaks at 6 and 8 minutes respectively. Pch has two conformations which elute at different time points as seen in the radio chromatogram (FIG. 1B). Free copper typically shows an elevated peak around 1 minute elution time, which is absent in this radio chromatogram, indicating almost all the copper-64 is bound to Pch within the sample. Extensive HPLC purity analysis involving varying concentrations of Pch was performed, ranging from 0.5, 1, 1.5, 2, 4, and 10 uL of 1 mg/mL Pch (FIGS. 1C-1G). Peaks were seen around 1 minute indicating high amounts of unbound, free copper with small peaks seen around 3.5 minutes. Further HPLC purity analysis involving varying incubation times of 10, 20, 30, 45, and 60 minutes shows multiple peaks at varying time points (FIGS. 2A-2E). This is most likely due to systemic error involving washing between sample runs and may not be evident of bound or unbound copper. This issue may also be resolved by running the sample for a longer period. Finally, UV chromatograms at 257 nm were obtained after incubating 25, 50, 100, and 200 uL of 1 mg/mL Pch to better understand the elution time relative to the concentration of Pch (FIGS. 3A-3E). This data shows that as Pch concentrations increase, the absorbance increases as well. Preliminary data also shows high complexation between Ga-68 and DFO at both pH 3 and pH 5.5 at 37° C. (Table 2).

TABLE 2
iTLC and HPLC characterization of DFO complexes.

| Radiometal | Temp | Time | pH 3 | pH 5.5 |
|---|---|---|---|---|
| ⁶⁸Ga | 37° C. | 20 mins | 93 | 62 |
| ⁶⁸Ga | 37° C. | 60 mins | 92 | 98 |
| ⁶⁴Cu | 37° C. | 20 mins | 1 | 4 |
| ⁶⁴Cu | 37° C. | 60 mins | 1 | 1 |

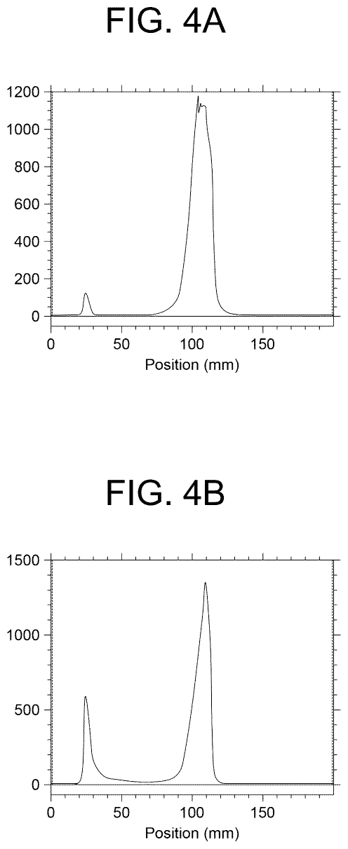

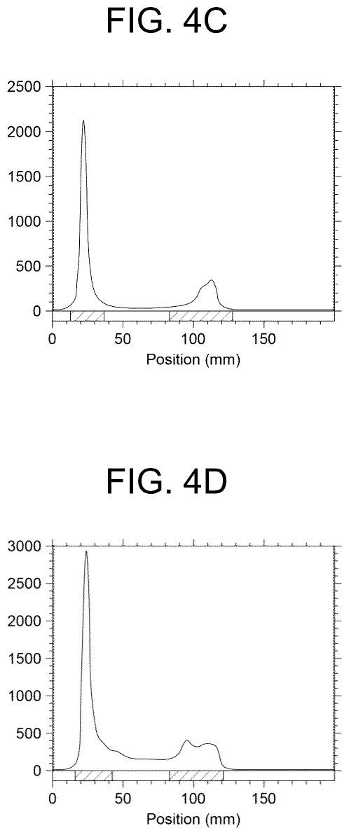

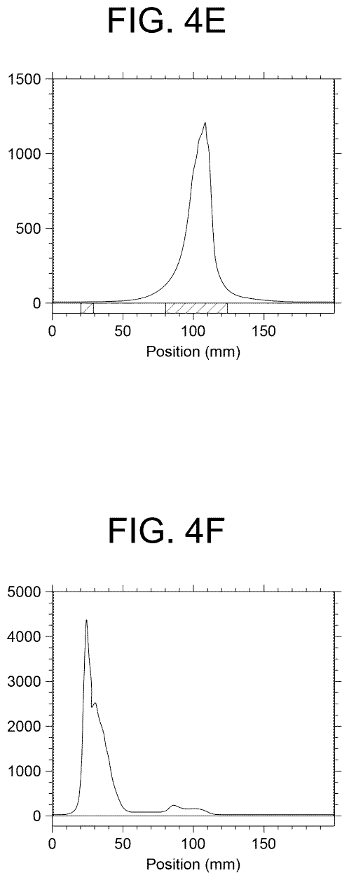

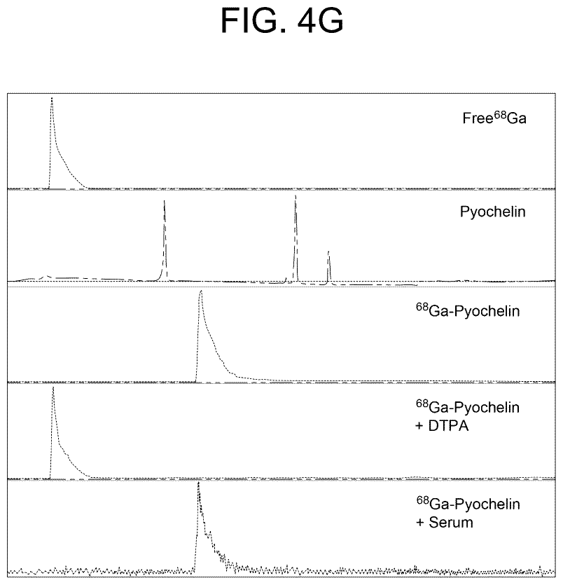

TLC analysis display mostly poor association between pyochelin and a multitude of radiometals consisting of lead-203, zirconium-89, titanium-45, manganese-52, as evident from the TLC graphs and UV chromatograms (FIGS. 4A-4E). All radiometals-pyochelin combinations were screened at pH 3.5, 5.5, and 7 at both 37° C. and 50° C. Only gallium-68 showed high complexation with pyochelin, with 100% complexation occurring at all three pH conditions at 37° C. (FIG. 4F). Additionally, radio chromatograms of gallium-64 bound Pch show zero free gallium-68 along with high stability in serum and were tested against diethylenetriamine pentaacetate (DTPA) to assess binding strength (FIG. 4G).

Quantitative values for pyochelin complexation with the listed radiometals at 37° C. are shown in Table 3.

TABLE 3
Quantitative values from TLC analysis between
pyochelin and different radiometals

| | pH 3.5 | pH 5.5 | pH 7 |
|---|---|---|---|
| Radiometal | (% complex) | (% complex) | (% complex) |
| Lead-203 | 3 | 3 | 2 |
| Zirconium-89 | 22.8 | 73.7 | 75.6 |
| Titanium-45 | 33.1 | 72.3 | 60 |
| Manganese-52 | 1.2 | 2.2 | 1.1 |
| Gallium-67 | 100 | 100 | 100 |

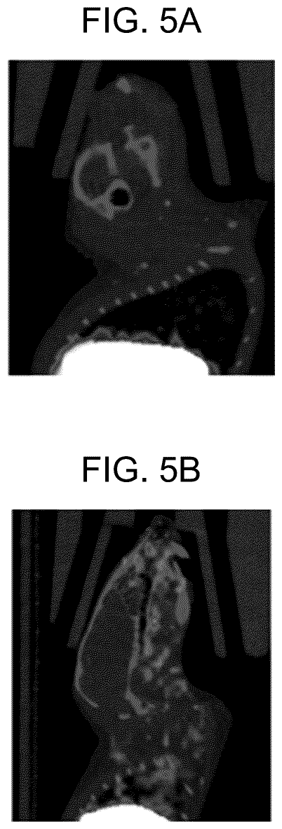

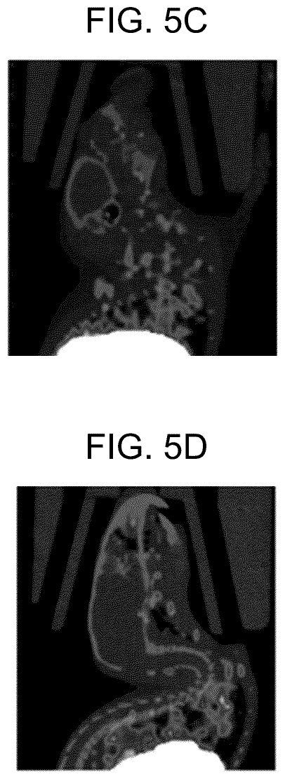

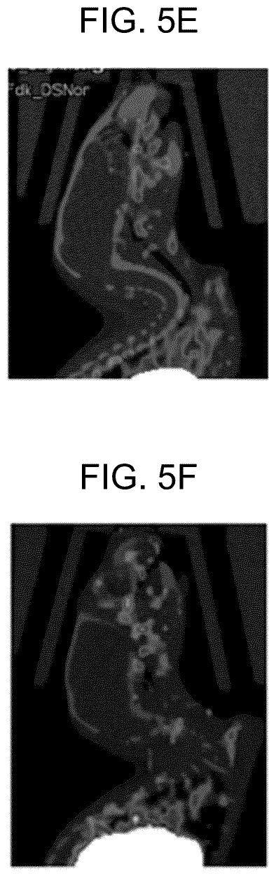

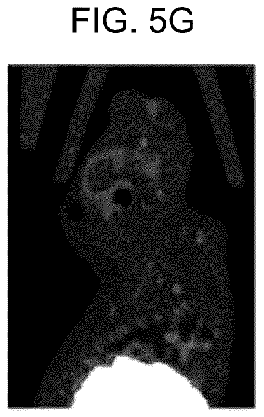

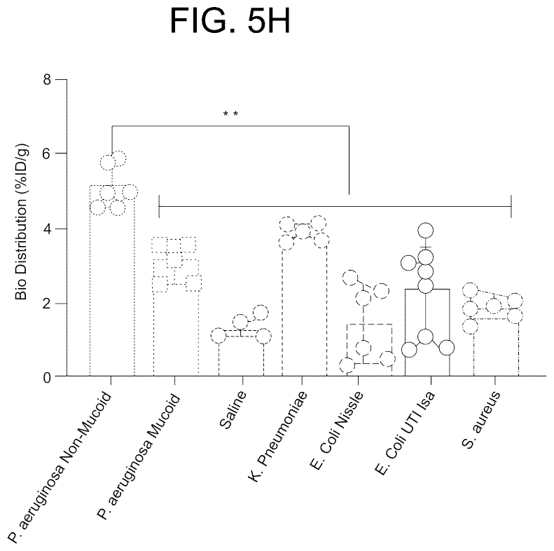

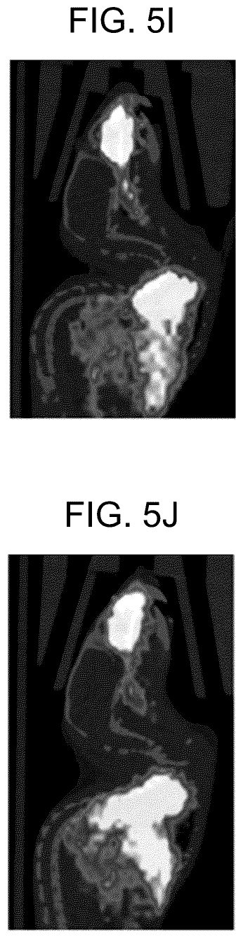

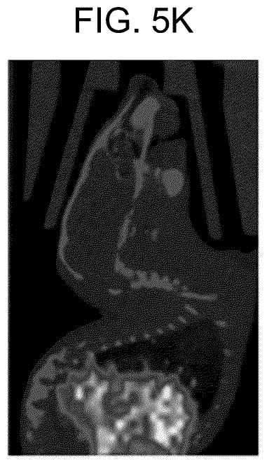

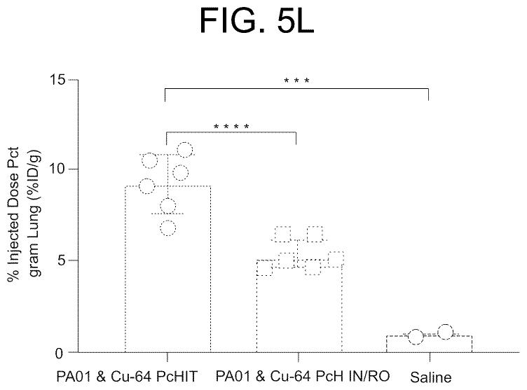

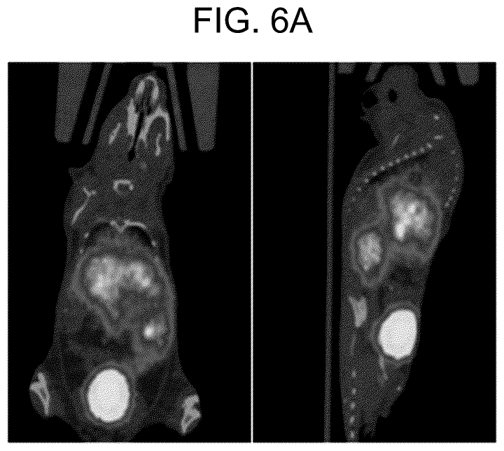

In vivo imaging data and the radioactive biodistribution data show an increased uptake in the Cu-64 Pch probe in the lungs in the fptA expressing P. aeruginosa with limited uptake in the non-fptA expressing organisms (FIGS. 5A-5H). The second in vivo study involving bacterial administration intra-tracheally shows a significant increase in biodistribution and probe accumulation compared to intra-nasal route of bacterial administration (FIGS. 51-5L).

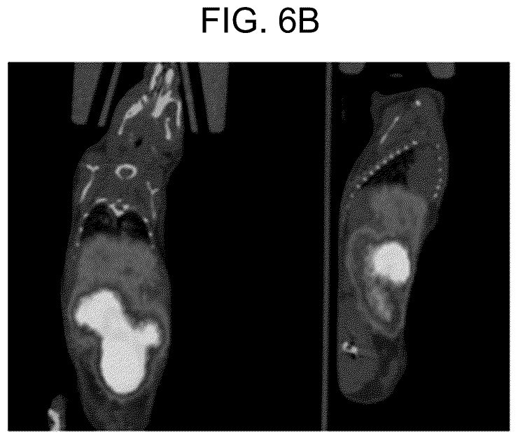

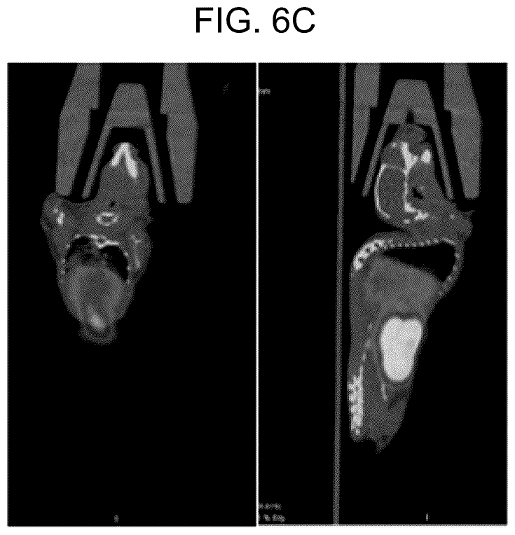

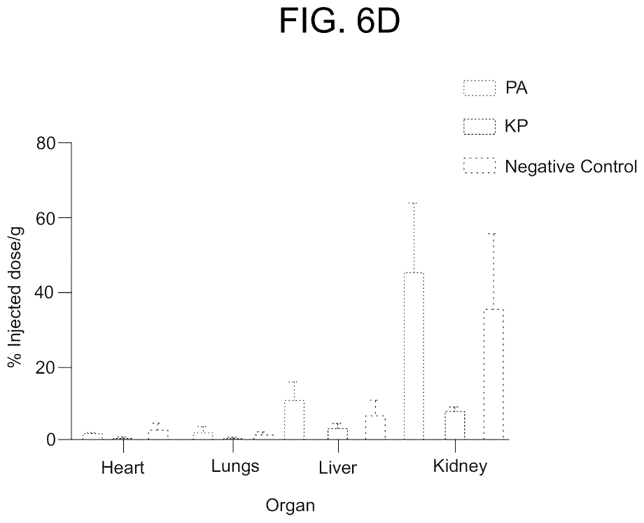

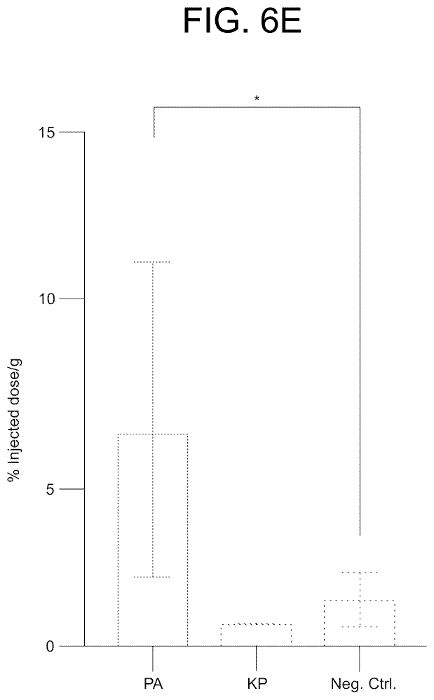

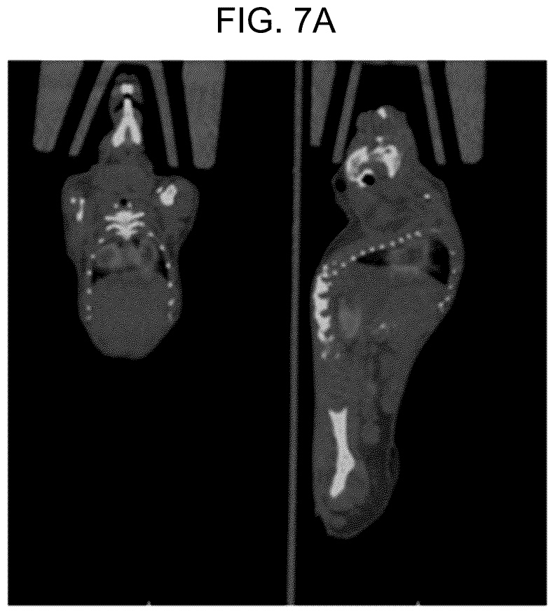

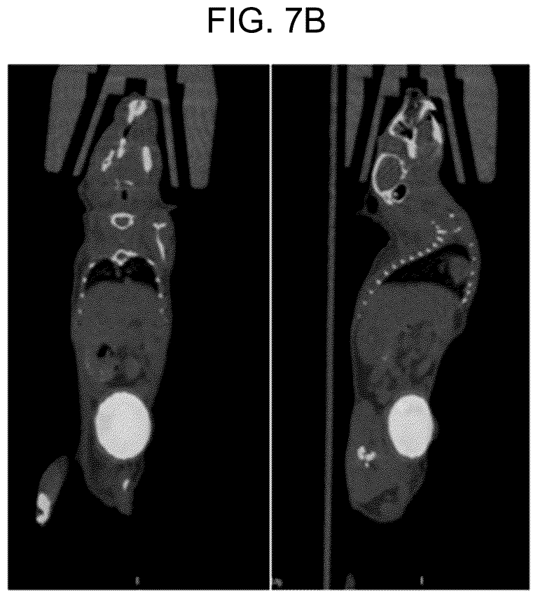

In vivo imaging and ex vivo biodistribution data for Ga-68 pyochelin show an increased uptake in the Ga-68 Pch probe in the lungs in the fptA expressing P. aeruginosa with limited uptake in the non-fptA expressing organisms (FIGS. 6A-6E).

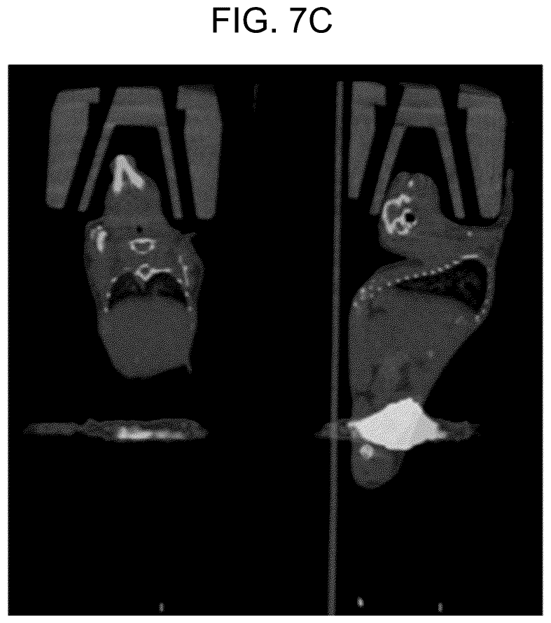

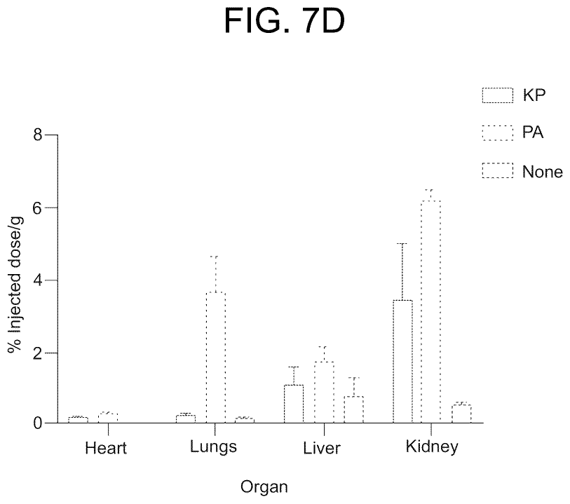

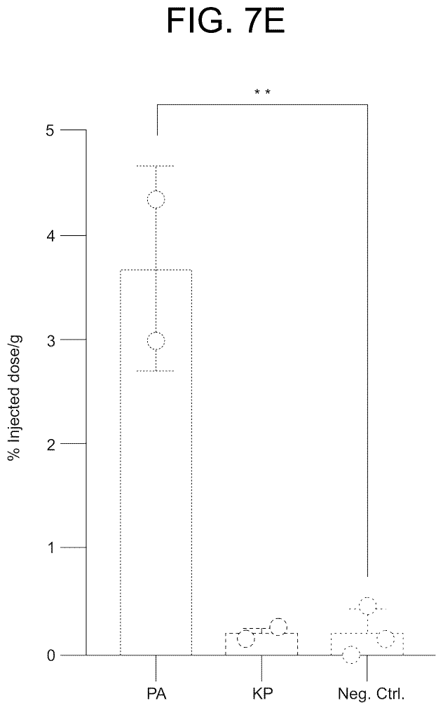

In vivo imaging and ex vivo biodistribution data for Ga-68 DFO show an increased uptake in the Ga-68 DFO probe in the lungs in the FoxA expressing P. aeruginosa with limited uptake in the non-FoxA expressing organisms (FIGS. 7A-7E).

### EXAMPLES

### Example 1

Preliminary in vitro data evaluating multiple siderophores with multiple radiometals to understand general binding characteristics was performed. To further evaluate Cu-64 binding affinity with Pch for in vivo applications, in vitro HPLC stability studies were performed to determine the percentage of bound Cu-64-Pch after incubation in serum. Samples were incubated for time points 0, 0.5, 1, 6, and 24. After incubation, samples were prepared for HPLC analysis by adding ˜500 uL of ammonium acetate with ethanol extraction solvent, spun at 12,000 RPM for 5 minutes. The top ⅔ of the supernatant was removed for HPLC analysis. After HPLC stability analysis, multiple HPLC purity studies were performed to determine the relative amount of free copper compared to Pch-bound copper. To prepare for purity analysis, ˜150 uCi of Cu-64 was incubated for 1 hour with varying amounts of 1 mg/mL pyochelin ranging from 0.5 to 10 uL, with ammonium acetate being added to reach a maximum volume of 350 uL. Additional purity studies were conducted using ˜150 uCi of Cu-64 along with 10 uL of 1 mg/mL pyochelin and ammonium acetate, but involved varying incubation periods consisting of 10, 20, 30, 45, and 60 minutes at 37° C. Lastly, UV chromatograms of Pch were generated using varying concentrations of Pch in ammonium acetate, consisting of 25, 50, 100, and 200 uL of Pch with ammonium acetate being added to reach 350 uL total volume. All HPLC analysis was conducted using 5-95% acetonitrile gradient over 10 minutes, with a flow rate of 1 mL/min using an Agilent Poroshell 120 EC-C18 column.

### Example 2

Extensive HPLC and thin-layer chromatography (TLC) analysis of multiple radiometals with pyochelin or DFO were performed. Radiometals were combined with siderophore at ratio of 20 uCi/ug in 50 uL buffer. Buffers consisted of sodium acetate (NaOAc), ammonium acetate, and HEPES at pH 3.5, 5.5, and 7 respectively. Complexation was heated to either 37° C. for one hour or 50° C. for one hour. Gallium-68 was prepared by concentrating the generator eluate in 0.1 M HCl onto a 30 mg Strata-XC cartridge, which was then air dried and the gallium eluted with three 150 uL aliquots of acetone. Reactions with gallium-68 were then carried out by adding 5 μg of siderophore with 50 uL of pH 3.5 sodium acetate and ˜250 uCi of gallium-68 radiometal and incubated at 37° C. for 20 minutes. Reaction products were placed on HPLC at 5-50% acetonitrile for 15 minutes using a kinetex evo C-18 column.

### Example 3

In vivo studies were performed using Balb/c mice. On day 1, Pseudomonas aeruginosa (PA01), Klebsiella pneumoniae, E. coli Nissle, E. coli UTI89, or Staphylococcus aureus bacterial concentrations of ˜3×10⁶ CFU/mL were prepared via centrifugation at 7 k RPM and washed three times with 1 mL of phosphate-buffered saline (PBS). 100 uL of bacterial concentrates were administered to groups of 3 mice intra-nasally, along with one group receiving 0.9% saline. After 24 hours, Cu-64 Pch was prepared and given retro-orbitally at a dose of ˜150 uCi per mouse. 48 hours after bacterial administration, the mice underwent PET/CT imaging. Immediately after imaging, the mice were sacked, and the lungs harvested for radioactive biodistribution measurements.

### Example 4

An additional in vivo study was performed using only PA01 bacteria. In this example, the bacteria were administered via intra-tracheal route, rather than intra-nasal. This route of administration is limited to 20 uL. Similarly, Ga-68 pyochelin studies were performed as follows: on day 1, PA01 was grown overnight to a concentration of ˜3×106 CFU/mL. After overnight incubation, the bacteria was washed with PBS and 100 uL was administered intranasally to each mouse. 5 hours after bacterial administration, Ga-68 was prepared and given retro-orbitally at a dose of ˜150-200uCi per mouse. 1 hour after Ga-68 injection, the mice were imaged via PET/CT, followed by euthanasia for biodistribution of the lungs.

While all the invention has been illustrated by a description of various embodiments, and while these embodiments have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

What is claimed is:

1. A method of targeted imaging of a bacterial infection in a subject comprising administering pyochelin (PcH) bound to a radiometal to the subject and imaging an area of the subject comprising the bacterial infection using a positron emission tomography-computed tomography (PET/CT) scan.

2. The method of claim 1 wherein the pyochelin bound to a radiometal is selected from the group consisting of copper-64 bound pyochelin and gallium-68 bound pyochelin.

3. The method of claim 1 wherein the pyochelin bound to a radiometal is copper-64 bound pyochelin.

4. The method of claim 3 wherein the pyochelin is bound to copper-64 in a reaction at a pH between 5.5 and 7.

5. The method of claim 1 wherein the pyochelin bound to a radiometal is gallium-68 bound pyochelin.

6. The method of claim 5 wherein the pyochelin is bound to gallium-68 in a reaction at a pH between 5.5 and 7.

7. The method of claim 1 wherein the bacterial infection comprises the bacteria Pseudomonas aeruginosa.

8. A method of targeted imaging of a bacterial infection in a subject comprising administering desferrioxamine B (DFO) bound to a radiometal to the subject and imaging an area of the subject comprising the bacterial infection using a positron emission tomography-computed tomography (PET/CT) scan.

9. The method of claim 8 wherein the DFO bound to a radiometal is selected from the group consisting of copper-64 bound DFO and gallium-68 bound DFO.

10. The method of claim 8 wherein the DFO bound to a radiometal is gallium-68 bound DFO.

11. The method of claim 10 wherein the DFO is bound to gallium-68 in a reaction at a pH between 3 and 7.

12. The method of claim 10 wherein the DFO is bound to gallium-68 in a reaction at a pH between 3 and 5.5.

13. The method of claim 8 wherein the DFO bound to a radiometal is copper-64 bound DFO.

14. The method of claim 13 wherein the DFO is bound to copper-64 in a reaction at a pH between 3 and 7.

15. The method of claim 8 wherein the bacterial infection comprises the bacteria Pseudomonas aeruginosa.
